(19) United States
(12) Patent Application Publication
(10) Pub. No.: US 2023/0103507 A1
(43) Pub. Date: Apr. 6, 2023

(54) METHOD FOR PREPARING 3,6-DICHLOROPYRAZINE-2-CARBONITRILE

(71) Applicant: NANJING CHEMPION BIOTECHNOLOGY CO., LTD., Nanjing (CN)

(72) Inventors: Jian CHEN, Nanjing (CN); Yongjiang BIAN, Nanjing (CN); Yaoqiang LI, Nanjing (CN)

(21) Appl. No.: 17/290,248

(22) PCT Filed: Aug. 11, 2020
(86) PCT No.: PCT/CN2020/108360

§ 371 Date: Apr. 29, 2021

(30) Foreign Application Priority Data
May 28, 2020 (CN) 202010481660.1

(51) Int. Cl. C07D 241/24

(52) U.S. Cl. CPC C07D 241/24

(57) ABSTRACT

The present disclosure provides a method for preparing a pyrazine compound of formula (I). In the present disclosure, 3-hydroxyl-6-bromopyrazine-2-amide is subjected to a reaction in the presence of phosphorus oxychloride and DIEA, during which adding an organic inorganic chloride makes the content of impurities greatly decreased, thereby obtaining 3,6-dichloropyrazine-2-carbonitrile with high-purity. 3,6-dichloropyrazine-2-carbonitrile prepared in the present disclosure is high in purity, low in production cost and suitable for industrial scale-up production and is very helpful for follow-on production of favipiravir API.

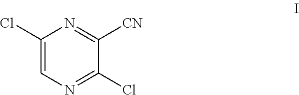

### CROSS REFERENCE TO RELEATED APPLICATION

This application claims priority of Chinese Patent Application No. 202010481660.1, filed on May 28, 2020 and titled “Method for Preparing 3,6-dichloropyrazine-2-carbonitrile”, the content of which is incorporated by reference herein in its entirety as part of the present application.

### TECHNICAL FIELD

The present disclosure relates to a method for preparing pyrazine compounds and belongs to the field of medicine and chemical synthesis.

### BACKGROUND ART

Favipiravir (API) serves as a compound that has remarkable effects on various viruses, particularly influenza viruses. Through study, it has been found that it exhibits good therapeutic activity on a novel coronavirus sars-cov-2. The structure formula of the API is shown as follows:

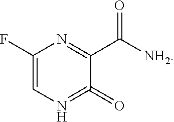

API is prepared mainly by the following steps: subjecting 3,6-dichloropyrazine-2-carbonitrile to a fluorination, and hydrolyzing the resulting fluorinated product, to obtain the API. In view of this, how to prepare the intermediate 3,6-dichloropyrazine-2-carbonitrile with low cost and excellent quality has become a major problem. At present, there are two major known routes for synthesizing 3,6-dichloropyrazine-2-carbonitrile, which are shown respectively as follows:

Route 1:

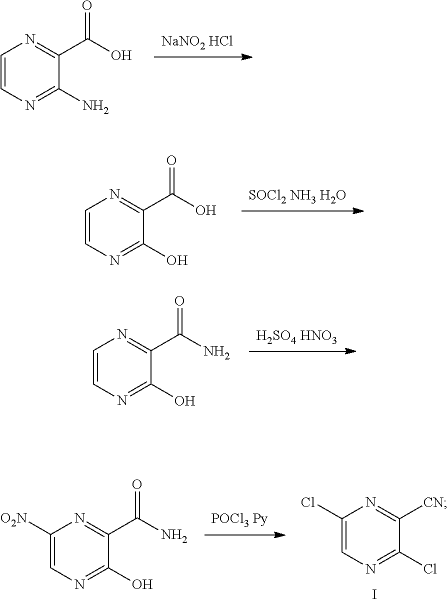

Route 2 (CN 102307865B):

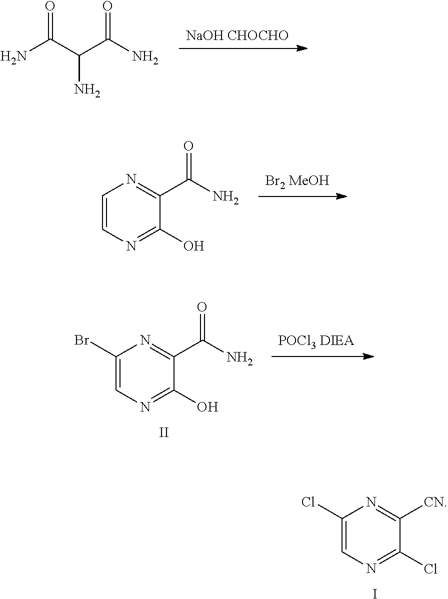

In terms of the cost and safety, Route 2 has greater advantages. However, during implementation, it is found that in the chlorination of 3 -hydroxyl-6-bromopyrazine-2-amide (II) to give 3,6-dichloropyrazine-2-carbonitrile (I), more impurities such as 3-bromo-6-chloropyrazine-2-carbonitrile (III) and 3-chloro-6-chloropyrazine-2-carbonitrile (IV) were produced, and they were difficult to be removed, thereby affecting the quality of the key intermediate 3,6-dichloropyrazine-2-carbonitrile. The reaction process is shown as follows:

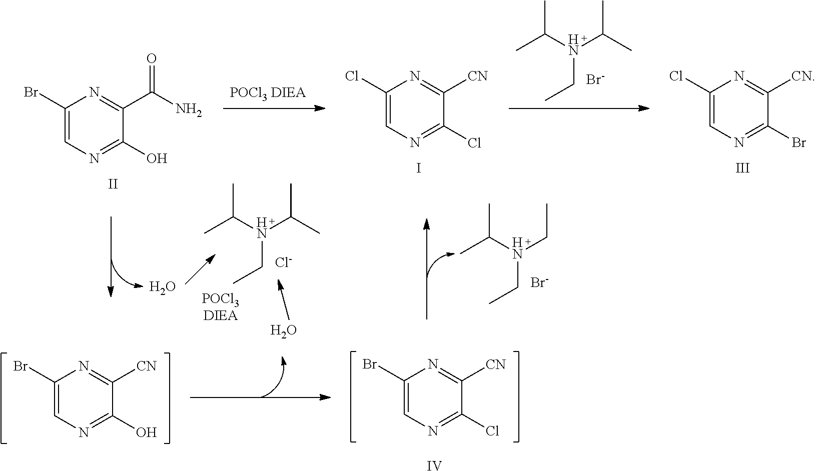

Among currently reported synthetic methods, there are no better methods for controlling production of the impurities such as 3-bromo-6-chloropyrazine-2-carbonitrile (III) and 3-chloro-6-chloropyrazine-2-carbonitrile (IV), which may greatly affect the quality of the key intermediate, thereby further affecting quality of the API. Therefore, there is an urgent need for an excellent reaction process for preparing the compound 3,6-dichloropyrazine-2-carbonitrile.

### SUMMARY

The present disclosure provides a method for preparing 3,6-dichloropyrazine-2-carbonitrile (I), in order to overcome the problem that there are more by-products such as 3-bromo-6-chloropyrazine-2-carbonitrile (III) and 3 -chloro-6-chloropyrazine-2-carbonitrile (IV) in the prior art.

The purpose of the present disclosure is to provide a method for preparing pyrazine compounds, which can greatly decrease bromine impurities and is suitable for industrial production.

Specifically, the present disclosure provides a method for preparing pyrazine compounds, comprising:

reacting 3-hydroxyl-6-bromopyrazine-2-amide with a chloride agent and an inorganic chloride at a temperature of 30° C. to 110° C. in the presence of a base with or without a solvent. The reaction equation is shown as follows:

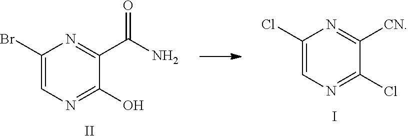

In some embodiments, the base is selected from the group consisting of triethylamine, diisopropylethylamine, N,N-dimethylaniline and N,N-diethylaniline, especially triethylamine or diisopropylethylamine, and further especially diisopropylethylamine.

In some embodiments, the chloride agent is selected from the group consisting of phosphorus oxychloride, thionyl chloride and phosphorus pentachloride, especially phosphorus oxychloride.

In some embodiments, the inorganic chloride is selected from the group consisting of lithium chloride, sodium chloride, potassium chloride, magnesium chloride and calcium chloride, especially lithium chloride or potassium chloride, and further especially lithium chloride.

In some embodiments, the reaction is carried out in the absence of solvent. Under the condition that the reaction is performed in the presence of a solvent, the solvent may be toluene and/or acetonitrile, especially toluene.

In some embodiments, the reaction is performed at a temperature of 30 to 110° C., especially 50 to 90° C.

In some embodiments, the base is used in an amount of 1.0 to 5.0 equivalent, especially 3.0 equivalent.

In some embodiments, the chloride agent is used in an amount of 3.0 to 6.0 equivalent, especially 4.0 equivalent.

In some embodiments, the inorganic chloride is used in an amount of 1.0 to 3.0 equivalent, especially 1.0 equivalent.

In the course of the study, it has been surprisingly found that, the addition of an inorganic chloride in the chlorination reaction system could increase the content of chloride ions in the system, so that 3-bromo-6-chloropyrazine-2-carbonitrile (III) and 3-chloro-6-chloropyrazine-2-carbonitrile (IV) could be effectively inhibited and decreased, thereby improving the quality of the key intermediate 3,6-dichloropyrazine-2-carbonitrile (I).

Compared with the prior art, the method according to the present disclosure makes it possible to significantly improve the quality of the key intermediate 3,6-dichloropyrazine-2-carbonitrile (I). Table 1 shows quality comparison of related substances to 3,6-dichloropyrazine-2-carbonitrile (I) prepared with or without the inorganic chloride.

TABLE 1
The quality comparison of related substances
to 3,6-dichloropyrazine-2-carbonitrile (I)

| | Without inorganic | With inorganic |
|---|---|---|
| Quality comparison | chloride | chloride |
| Purity of | 92.59% | 98.33% |
| 3,6-dichloropyrazine-2- | | |
| carbonitrile (I) | | |
| Content of impurity | 1.13% | 0.36% |
| 3-bromo-6-chloropyrazine-2- | | |
| carbonitrile (III) | | |
| Content of impurity | 6.16% | 1.15% |
| 3-chloro-6-chloropyrazine-2- | | |
| carbonitrile (IV) | | |

According to the above contents of the present disclosure, through general technical knowledge and conventional means in the art, many other modifications, replacements or changes may be made without departing from the basic technical concept of the present disclosure.

### DETAILED DESCRIPTION

The above contents of the present disclosure will be further described in detail below by specific embodiments in the form of examples. However, it should be understood that, the scope of the present disclosure is not only limited to examples below. All technologies achieved based on the above contents of the present disclosure should fall within the scope of the present disclosure.

Raw materials and equipment used in specific examples of the present disclosure are all known products that are commercially available.

### EXAMPLE 1: SYNTHESIS OF 3,6-DICHLOROPYRAZINE-2-CARBONITRILE

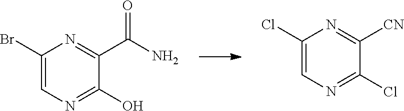 [SMILES: NC(=O)c1nc(Br)cnc1O ; N#Cc1nc(Cl)cnc1Cl]

3-hydroxyl-6-bromopyrazine-2-amide (10 g) was mixed with lithium chloride (1.94 g) and phosphorus oxychloride (28 g), and then stirred and raised to a temperature of 50° C. After that, diisopropylethylamine (17.78 g) was added thereto, and the resulting mixture was raised to a temperature of 80° C. and then stirred for 1 hour. Later, the mixture was cooled to a temperature of about 30° C. and was slowly added into ice water for quenching, and then filtered to obtain a filter cake. The filter cake was pulped with isopropanol (15 mL), obtaining 3,6-dichloropyrazine-2-carbonitrile (6.6 g, light yellow solid).

### EXAMPLE 2: SYNTHESIS OF 3,6-DIFLUOROPYRAZINE-2-CARBONITRILE

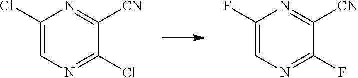 [SMILES: N#Cc1nc(Cl)cnc1Cl ; N#Cc1nc(F)cnc1F]

3,6-dichloropyrazine-2-carbonitrile (10 g) was added into DMF (60 mL), and TBAF (in an amount of catalytic amount) and potassium fluoride (20 g) were added thereto. The resulting mixture was raised to a temperature of 60° C. and reacted for 12 hours. After the reaction was ended, the mixture was cooled to ambient temperature, and added into water for quenching, to obtain an aqueous phase and an organic phase. The aqueous phase was extracted with methyl tert-butyl ether (50 mL) for three times, and the organic phase was combined. The combined organic phase was washed with water (50 mL), dried and concentrated, obtaining 3,6-difluoropyrazine-2-carbonitrile (crude, which was directly used for the subsequence reaction without further purifying).

### EXAMPLE 3: SYNTHESIS OF 3-HYDROXYL-6-FLUOROPYRAZINE-2-CYANO

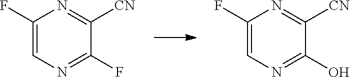 [SMILES: N#Cc1nc(F)cnc1F ; N#Cc1nc(F)cnc1O]

3,6-difluoropyrazine-2-carbonitrile (crude, 7 g) obtained in example 2 was added into DMF (30 mL), then the mixture was cooled with an ice water bath. Then acetic acid (6 g) and triethylamine (10 g) was added thereto in sequence. After the completion of adding, the mixture was heated and stirred overnight. After the reaction was ended, the resulting mixture was added into ice water, and pH value thereof was adjusted to 3-4. Then the mixture was extracted with methyl tert-butyl ether (100 mL) to obtain an aqueous phase and an organic phase. The organic phase was dried with anhydrous sodium sulfate, filtered and concentrated to obtain a crude product. Then the crude product was pulped with n-heptane obtaining 3-hydroxyl-6-fluoropyrazine-2-cyano (6 g, pale brown solid).

### EXAMPLE 4: SYNTHESIS OF 3-HYDROXYL-6-FLUOROPYRAZINE-2-AMIDO

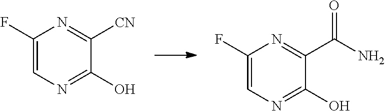 [SMILES: N#Cc1nc(F)cnc1O ; NC(=O)c1nc(F)cnc1O]

3-hydroxyl-6-fluoropyrazine-2-cyano (6 g) obtained in example 3 was added into an aqueous NaOH solution, then the mixture was cooled to a temperature of 1 to 10° C. hydrogen peroxide was added dropwise thereto. After completion of adding hydrogen peroxide, the mixture was slowly returned to ambient temperature and continuously stirred for 6 hours. After the reaction was ended, the mixture was adjusted to have a pH value of 3 to 4 with hydrochloric acid, and then filtered to obtain a filter cake. The filter cake was leached with purified water, and the leached filter cake was collected and dried in vacuum, obtaining 3-hydroxyl-6-fluoropyrazine-2-amido (5.5 g, white solid).

The above descriptions are merely preferred embodiments of the present disclosure. Equivalent changes and modifications made in accordance with the scope of the present disclosure should fall within the scope of the present disclosure.

1. A method for preparing a pyrazine compound of formula (I), comprising,
reacting 3-hydroxyl-6-bromopyrazine-2-amide with a chloride agent and an inorganic chloride at a temperature of 30 to 110° C. in the presence of a base with or without a solvent,

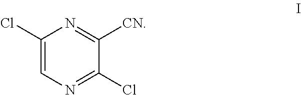

2. The method according to claim 1, wherein the base is selected from the group consisting of triethylamine, diisopropylethylamine, N,N-dimethylaniline and N,N-diethylaniline.

3. The method according to claim 2, wherein the base is selected from the group consisting of triethylamine and diisopropylethylamine.

4. The method according to claim 3, wherein the base is diisopropylethylamine.

5. The method according to claim 1, wherein the base is used in an amount of 1.0-5.0 equivalent.

6. The method according to claim 1, wherein the chloride agent is selected from the group consisting of phosphorus oxychloride, thionyl chloride and phosphorus pentachloride.

7. The method according to claim 6, wherein the chloride agent is phosphorus oxychloride.

8. The method according to claim 1, wherein the chloride agent is used in an amount of 3.0-6.0 equivalent.

9. The method according to claim 1, wherein the inorganic chloride is selected from the group consisting of lithium chloride, sodium chloride, potassium chloride, magnesium chloride and calcium chloride.

10. The method according to claim 9, wherein the inorganic chloride is selected from the group consisting of lithium chloride and potassium chloride.

11. The method according to claim 10, wherein the inorganic chloride is lithium chloride.

12. The method according to claim 1, wherein the inorganic chloride is used in an amount of 1.0-3.0 equivalent.

13. The method according to claim 1, wherein the solvent is toluene or acetonitrile.

14. The method according to claim 13, wherein the solvent is toluene.

15. The method according to claim 1, wherein the reacting is performed at a temperature of 30 to 110° C.

16. The method according to claim 15, wherein the reacting is performed at a temperature of 50 to 90° C.
